(19) United States
(12) Patent Application Publication
(10) Pub. No.: US 2026/0355178 A1
(43) Pub. Date: Nov. 12, 2026

(54) METHOD OF TREATING ADULT-ONSET AUTOSOMAL DOMINANT LEUKODYSTROPHY (ADLD)

(71) Applicant: AGENCY FOR SCIENCE, TECHNOLOGY AND RESEARCH, Singapore (SG)

(72) Inventors: Mattheus Xing Rong FOO, Singapore (SG); Brian Edmund BURKE, Singapore (SG); Oliver DREESEN, Singapore (SG); Peh Fern (Wang Peifen) ONG, Singapore (SG)

(21) Appl. No.: 19/122,517

(22) PCT Filed: Sep. 29, 2023
(86) PCT No.: PCT/SG2023/050653

§ 371 Date: Apr. 18, 2025

(30) Foreign Application Priority Data
Oct. 21, 2022 (SG) 10202251453E

(51) Int. Cl. A61K 31/4709; A61K 31/216; A61K 31/4545; A61P 25/28

(52) U.S. Cl. CPC A61K 31/4709; A61K 31/216; A61K 31/4545; A61P 25/28

(57) ABSTRACT

The present invention relates to methods of treating diseases or conditions associated with abnormal levels of lamin B1 protein. Disclosed herein are methods of treating pathologies of lamin B1 accumulation, such as Adult-onset Autosomal Dominant Leukodystrophy (ADLD), using farnesyltransferase inhibitors (FTIs), such as FTI-277, lonafarnib or Tipifarnib.

### TECHNICAL FIELD

The present invention generally relates to methods of treating diseases or conditions associated with abnormal levels of lamin B1 protein. In particular, disclosed herein are methods of treating pathologies of lamin B1 accumulation, such as Adult-onset Autosomal Dominant Leukodystrophy (ADLD), using farnesyltransferase inhibitors (FTIs).

### BACKGROUND

Perturbations or mutations in the nuclear lamina and its components cause a variety of different human diseases including cardiomyopathies, muscular dystrophies, lipodystrophies and premature aging syndromes, collectively called laminopathies. The majority of these diseases are caused by mutations in the lamin A gene (LMNA), though overexpression of the lamin B1 gene (LMNB1) is also associated with certain pathologies. Lamin A, B1 and B2 undergo post-translational modifications by addition of a farnesyl moiety to their C-terminal cysteine residue. In the case of lamin A, the 15 C-terminal amino acids, including the farnesylated tail, are subsequently cleaved, thereby forming the mature lamin A protein. In contrast, both lamin B1 and B2 retain their farnesyl group.

Adult-onset autosomal dominant leukodystrophy (ADLD) is a slowly progressive neurological disorder characterized by demyelination in the central nervous system and age-dependent motor deficit. It is a rare genetic disorder which is caused by a duplication of the LMNB1 locus which results in elevated lamin B1 protein levels. Small fluctuations in lamin B1 expression can have remarkable molecular and functional consequences for cells of the nervous system, especially the CNS. Patients usually start to exhibit symptoms, which include motor and sensory dysfunction, in their mid-30s. There is currently no effective treatment available for ADLD or other laminopathies associated with abnormal levels of lamin B1. The contribution of lamin B1 farnesylation to the development of these diseases is also unknown.

Accordingly, it would be desirable to overcome or ameliorate at least one of the above-described problems.

### SUMMARY

Disclosed herein is a method of inhibiting farnesylation of lamin B1 protein in a cell from a subject, the method comprising administering to the cell an effective amount of a farnesyltransferase inhibitor (FTI).

Disclosed herein is a method of inhibiting accumulation of lamin B1 protein in a cell from a subject, the method comprising administering to the cell an effective amount of a farnesyltransferase inhibitor (FTI).

Disclosed herein is a method of treating a disease or condition in a subject associated with abnormal levels of lamin B1 protein, the method comprising administering an effective amount of a farnesyltransferase inhibitor (FTI) to the subject.

Disclosed herein is a farnesyltransferase inhibitor for use in treating a disease or condition in a subject associated with abnormal levels of lamin B1 protein.

Disclosed herein is the use of a farnesyltransferase inhibitor in the manufacture of a medicament for treating a disease or condition in a subject associated with abnormal levels of lamin B1 protein.

Disclosed herein is a method of treating Adult-onset Autosomal Dominant Leukodystrophy (ADLD) in a subject, the method comprising administering an effective amount of a farnesyltransferase inhibitor (FTI) to the subject.

Disclosed herein is a farnesyltransferase inhibitor for use in treating Adult-onset Autosomal Dominant Leukodystrophy (ADLD) in a subject.

Disclosed herein is the use of a farnesyltransferase inhibitor in the manufacture of a medicament for treating Adult-onset Autosomal Dominant Leukodystrophy (ADLD) in a subject.

### BRIEF DESCRIPTION OF THE DRAWINGS

Embodiments of the present invention will now be described, by way of non-limiting example, with reference to the drawings in which:

FIG. 1 shows that genetically preventing farnesylation of lamin B1 prevents its accumulation in human fibroblasts. Wild type v5-LB1 CAIM or a non-farnesylatable mutant of lamin B1 (v5-LB1 SAIM) were expressed (upon DOX addition) in normal (WT) or lamin A/C (shLA/C) human fibroblasts. Western blot shows reduced accumulation of v5LB1 SAIM in both WT and shLA/C fibroblasts. Actin is used as loading control.

FIG. 2 shows that treatment with 3 different FTIs prevents lamin B1 accumulation. (A) Schematic representation of experimental designs for panels (B) and (C) using an inducible LB1 NDF cell line. (B) Representative western blot of NDFs expressing LB1 over 3 days in the presence of DMSO control, FTI-277, Lonafarnib and Tipifarnib. v5, GAPDH, LA/C, hours post DOX-induction are as indicated. (C) Quantification of 3 independent sets represented in (B). Protein levels were normalised against GAPDH and relative to its 8 h v5 levels of DMSO control (n=3, Two-way ANOVA with Bonferroni's post-test, *=<0.05, **=<0.01, ***=<0.001, ****=<0.0001).

FIG. 3 shows doxycycline-inducible expression of lamin B1: with DMSO or lovastatin over the course of 72 hours.

FIG. 4 shows that FTI-277 specifically reduces LB1 accumulation. (A) Schematic of both LB1 constructs expressed under a DOX-inducible promoter (LB1 and LB1 SAIM). The SAIM mutation prevents the farnesylation of LB1 through the substitution of the cysteine in the CaaX box. (B) Schematic representation of experimental designs for panels (C) and (D) using the cell lines indicated in (A). (C) Representative western blot of NDFs expressing LB1 or LB1 SAIM over 16 days in the presence of DMSO control or FTI-277. v5, GAPDH, LA/C, days post DOX-induction are as indicated. (D) Quantification of 3 independent sets represented in (C). Protein levels were normalised against GAPDH and relative to its day 4 v5 levels of DMSO control (n=3, Two-way ANOVA with Bonferroni's post-test, *=<0.05, **=<0.01, ***=<0.001).

### DETAILED DESCRIPTION

The present specification teaches methods of inhibiting farnesylation of lamin B1 protein. Disclosed herein is a method of inhibiting farnesylation of lamin B1 protein in a cell from a subject, the method comprising administering to the cell an effective amount of a farnesyltransferase inhibitor (FTI). In one embodiment, inhibition of farnesylation of lamin B1 inhibits accumulation of lamin B1 protein in the cell. In another embodiment, inhibition of farnesylation of lamin B1 reduces lamin B1 levels in the cell.

Without being bound by theory, the inventors have discovered that genetically preventing lamin B1 farnesylation slows the intracellular accumulation of the protein and can be used to treat lamin B1-associated diseases like adult-onset autosomal dominant leukodystrophy (ADLD). The inventors have further discovered that farnesyltransferase inhibitors (FTIs) can be used to prevent lamin B1 accumulation. Hence, FTIs can be used to address diseases caused by abnormally elevated levels of lamin B1 or other permanently farnesylated proteins.

Also disclosed herein is a method of inhibiting accumulation of lamin B1 protein in a cell from a subject, the method comprising administering to the cell an effective amount of a farnesyltransferase inhibitor (FTI).

Also disclosed herein is a method of reducing the level of lamin B1 protein in a cell from a subject, the method comprising administering to the cell an effective amount of a farnesyltransferase inhibitor (FTI).

In one embodiment, the subject is suffering from a disease or condition associated with abnormal levels of lamin B1 protein. Such a disease or condition would generally be characterised by an abnormal intracellular level of lamin B1 protein. For the avoidance of doubt, the abnormal levels of lamin B1 need not be a causative factor for the disease or condition and may be, for example, secondary to other genetic and/or cellular defects also present in the disease or condition. The term “associated with” includes an increased risk of developing the disease or condition as well as the disease or condition itself. Thus, for instance, an abnormal increase in lamin B1 levels may be associated with ADLD and the tendency to develop ADLD.

The term “abnormal level” refers a chronic or progressive change in the level of a protein which results in a disease or pathological condition. An abnormal level of a protein may be determined by comparison to a normal or reference level. This normal or reference level can be found in a control, a standard, a population standard, etc. For instance, where the abnormal protein level is associated with a disease, the normal or reference level may be the protein level in a cell (or the average protein level in a population of cells) from an individual who is not suffering from the disease, or a level that is the population standard of individuals believed not to be suffering from the disease, etc.

The abnormal level may be an abnormally high or abnormally low level of lamin B1. Lamin B1 levels may be abnormal in one or more cell types, tissues or organs in a subject.

The terms “elevated level” and “accumulation” are used interchangeably herein to refer to levels of a protein that are abnormally high. The level of lamin B1 may be abnormally high if it is above a reference level by, for example, at least about 10%, at least about 20%, at least about 30%, at least about 40%, at least about 50%, at least about 60%, at least about 70%, at least about 80%, at least about 90%, at least about 100%, at least about 2.5-fold, at least about 3-fold, at least about 3.5-fold, at least about 4-fold, at least about 4.5-fold, at least about 5-fold, or more.

The terms “decreased level” and “reduction” are used interchangeably herein to refer to levels of a protein that are abnormally low. The level of lamin B1 may be abnormally low if it is below a reference level by, for example, at least about 10%, at least about 20%, at least about 30%, at least about 40%, at least about 50%, at least about 60%, at least about 70%, at least about 80%, at least about 90%, or about 100%.

Abnormal lamin B1 levels may be due to an abnormal nucleic acid, such as an abnormal LMNB1 nucleic acid encoding lamin B1. Such abnormality includes but is not limited to: (1) a mutation in the nucleic acid, such as a point mutation (e.g., a single nucleotide polymorphism) or deletion, duplication, addition or inversion of a stretch of nucleotides; (2) a mutation in the control sequence(s) associated with that nucleic acid such that replication or expression of the nucleic acid is altered (such as the functional inactivation of a promoter); (3) a change in the amount or copy number of the nucleic acid in a cell as compared to a control or standard (such as a duplication or genomic amplification of the nucleic acid, or overexpression of the mRNA); and (4) an alteration in a sequence that controls the splicing mechanism, in such a way that either a normal splice signal is inactivated or an abnormal splice signal is created. It will be understood that these types of abnormalities can co-exist in the same nucleic acid or in the same cell; for instance, a genomic-amplified nucleic acid sequence may also contain one or more point mutations. In addition, it is understood that an abnormality in a nucleic acid may be associated with, and in fact may cause, an abnormality in expression of the corresponding protein.

Abnormal lamin B1 levels may also be due to abnormal protein expression. This includes but is not limited to: (1) a mutation in the protein such that one or more of the amino acid residues is different; (2) a deletion, addition or duplication of a stretch of amino acid in the sequence of the protein; (3) expression of an increased or decreased amount of the protein as compared to a control or standard; (4) alteration of the subcellular localization or targeting of the protein; (5) alteration of the temporally regulated expression of the protein (such that the protein is expressed when it normally would not be, or alternatively is not expressed when it normally would be); and (6) alteration of the localized (e.g., organ-or tissue-specific) expression of the protein (such that the protein is not expressed where it would normally be expressed or is expressed where it normally would not be expressed), each compared to a control or standard.

Another type of protein abnormality that is contemplated herein involves changes in the post-translational processing of a protein. A protein that is abnormally post-translationally processed includes, for instance, proteins that are processed in a different way, or to a different extent, than the wild-type (normal) version of the protein. For instance, wild-type lamin B1 protein is permanently or constitutively farnesylated, and a lamin B1 protein that lacks a farnesyl group (e.g., lamin B1 lacking a farnesylation site) may be considered an abnormally farnesylated form of lamin B1. Other potential abnormal post-translational processing includes changes in other added groups, e.g., methylations, phosphorylations, glycosylations, and so forth.

In one embodiment, the subject is suffering from a disease or condition associated with an accumulation of lamin B1 protein. Non-limiting examples of diseases or conditions associated with an accumulation of lamin B1 include Adult-onset Autosomal Dominant Leukodystrophy (ADLD), Werner Syndrome, ataxia telangiectasia, Huntington's Disease, and cancer, including but not limited to pancreatic, liver and ovarian cancer.

In one embodiment, the subject is suffering from Adult-onset Autosomal Dominant Leukodystrophy (ADLD). ADLD is characterized by progressive demyelination in the brain and spinal cord of subjects, leading to autonomic nervous system dysfunction in adulthood and eventual movement impairment. It is a genetic disease caused by duplications in the lamin B1 gene (LMNB1) or deletions near the LMNB1 locus which result in an excess of intracellular lamin B1. FTIs are thus particularly suited to treating ADLD by inhibiting accumulation of lamin B1.

As used herein, the term “subject” refers to a mammal. A subject can be a human or a non-human mammal such as a dog, cat, bovid, equine, mouse, rat, rabbit, or transgenic species thereof. The subject can be a patient. In one embodiment, the subject is a human.

The cell may be any cell expressing lamin B1 protein, including but not limited to cells of the central and peripheral nervous system, musculoskeletal system, digestive system, cardiovascular system, lymphatic system, respiratory system, integumentary system, digestive system, male and female reproductive systems, endocrine system, urinary systems.

In one embodiment, the cell is a cell of the central or peripheral nervous system. The cell may be, for example, a neuron, oligodendrocyte, astrocyte, ependymal cell, microglial cell, Schwann cell, satellite cell, or an immune or vascular cell of the central or peripheral nervous system. The cell may be involved in creating a myelin sheath around neurons, for example, an oligodendrocyte or astrocyte in the central nervous system, or a Schwann cell in the peripheral nervous system.

In one embodiment, the cell has abnormal levels of lamin B1 protein, which may be an abnormally high or abnormally low level of lamin B1. In one embodiment, the cell has an accumulation of lamin B1 protein.

As used herein, a “farnesyltransferase inhibitor (FTI)” refers to a substance which results in a decrease in farnesyltransferase expression or activity. The reduction in farnesyltransferase expression or activity may be specific to a cell, tissue or organ, and it may be a transient or permanent decrease.

Farnesyltransferase is an enzyme that transfers a farnesyl group from farnesyldiphosphate to the cysteine residue of a CAAX box motif in a protein (C: cysteine, A: an aliphatic amino acid, X: any amino acid). The enzyme is a heterodimer composed of two subunits, an α-subunit encoded by the FNTA gene, and a β-subunit encoded by the FNTB gene. The α-subunit is also found in geranylgeranyltransferases and is required for structural stability of the farnesyltransferase enzyme. The β-subunit is responsible for substrate binding and catalysis. By way of non-limiting example, an FTI may target one or both enzyme subunits, the interaction between the subunits, or the interaction between the enzyme and a non-substrate protein or cellular or subcellular component.

Substances that result in a decrease in farnesyltransferase activity include but are not limited to small molecules, peptides (e.g., allosteric antagonists or substrate mimetics), proteins (e.g., antibodies), nucleic acids (e.g., aptamers) and conjugates of two or more of such. The inhibitor may be a nucleic acid encoding an inhibitory peptide or polypeptide. The inhibitor may inhibit enzyme activity directly (e.g., by preventing interaction with a substrate or by altering the structure of the enzyme) or indirectly (e.g., by affecting the subcellular localization of the enzyme or enzyme interaction with a cofactor or subunit).

Substances that inhibit farnesyltransferase expression may act on the LMNB1 gene (e.g., by selectively altering the nucleic acid sequence of the gene to prevent gene expression and/or produce a non-functional enzyme), on a promoter operably-linked to the LMNB1 gene (e.g., to prevent or reduce transcription of the gene), or on an RNA product of the LMNB1 gene (e.g., by degrading or silencing lamin B1 mRNA). The inhibitor may inhibit gene transcription and/or translation from an RNA product of the gene, induce exon skipping or otherwise interfere with RNA splicing, or mediate sequence-specific cleavage, degradation or editing of LMNB1 or an RNA product of the gene. The inhibitor may be a nucleic acid molecule, e.g., a single-stranded or double-stranded DNA or RNA molecule. The inhibitor may mediate inhibition on its own or alongside a polypeptide or another nucleic acid molecule. Furthermore, the inhibitor may undergo biochemical or enzymatic processing after administration (for example, in a cell or tissue of the subject) to attain a functional inhibitory state.

In one embodiment, the FTI inhibits farnesyltransferase expression. In one embodiment, the FTI is a nucleic acid inhibitor. Non-limiting examples of nucleic acid inhibitors include antisense compounds (e.g., antisense oligonucleotides (ASOs), gapmers and the like); nucleic acid molecules mediating RNA interference, including but not limited to short hairpin RNA (shRNA), small interfering RNA (siRNA) and variants and precursors of such (e.g., small segmented siRNA, small interfering ribonucleic neutrals, Dicer substrate siRNA, etc.), and microRNA (miRNA) and precursors; and guide RNAs (gRNAs) mediating sequence-specific base editing, gene or RNA editing, or DNA or RNA cleavage in association with a Cas protein. The inhibitor may be a vector (e.g., plasmid vectors, viral vectors, etc.) encoding a nucleic acid inhibitor.

The terms “polynucleotide”, “genetic material”, “genetic forms”, “nucleic acids” and “nucleotide sequence” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art.

The term “nucleotide” refers to a ribonucleotide or a deoxyribonucleotide or modified form thereof, as well as an analogue thereof. Nucleotides include species that comprise purines, e.g., adenine, hypoxanthine, guanine, and their derivatives and analogues, as well as pyrimidines, e.g., cytosine, uracil, thymine, and their derivatives and analogues.

Nucleotide analogues include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogues are also meant to include nucleotides with bases such as inosine, queuosine, xanthine, sugars such as 2′-methyl ribose, non-natural phosphodiester linkages such as methylphosphonates, phosphorothioates and peptides.

Modified bases refer to nucleotide bases such as, for example, adenine, guanine, cytosine, thymine, uracil, xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogues thereof that are not ribosyl. For example, the sugar moieties may be, or be based on, mannoses, arabinoses, glucopyranoses, galactopyranoses, 4′-thioribose, and other sugars, heterocycles, or carbocycles.

In one embodiment, the FTI inhibits farnesyltransferase activity. The FTI may be a small molecule. Non-limiting examples of FTIs which inhibit farnesyltransferase activity include chaetomellic acid A, clavaric acid, FPT Inhibitor I, FPT Inhibitor II, FPT Inhibitor III, FTase Inhibitor I, FTase Inhibitor II, FTI-276 trifluoroacetate salt, FTI-277 trifluoroacetate salt, GGTI-297, L-744,832 dihydrochloride, manumycin A, gingerol, gliotoxin, a-hydroxy farnesyl phosphonic acid, tipifarnib, lonafarnib (SCH-66336), CP-609,754, BMS-214662, L778123, L744823, L739749, R208176, AZD3409 or FTI-277. In one embodiment, the FTI is FTI-277, lonafarnib or Tipifarnib.

In some embodiments, the FTI reduces farnesylation of lamin B1 by at least about 10%. The FTI may reduce farnesylation of lamin B1 by at least about 10%, at least about 20%, at least about 30%, at least about 40%, at least about 50%, at least about 60%, at least about 70%, at least about 80%, at least about 90%, or about 100%. The FTI may reduce farnesylation of lamin B1 by about 10%, about 20%, about 30%, about 40%, about 50%, about 60%, about 70%, about 80%, about 90% or about 100%.

In some embodiments, the FTI reduces accumulation of lamin B1 by at least about 10%. The FTI may reduce accumulation of lamin B1 by at least about 10%, at least about 20%, at least about 30%, at least about 40%, at least about 50%, at least about 60%, at least about 70%, at least about 80%, at least about 90%, or about 100%. The FTI may reduce accumulation of lamin B1 by about 10%, about 20%, about 30%, about 40%, about 50%, about 60%, about 70%, about 80%, about 90% or about 100%.

In some embodiments, the FTI reduces lamin B1 levels by at least about 10%. The FTI may reduce lamin B1 levels by at least about 10%, at least about 20%, at least about 30%, at least about 40%, at least about 50%, at least about 60%, at least about 70%, at least about 80%, at least about 90%, or about 100%. The FTI may reduce lamin B1 levels by about 10%, about 20%, about 30%, about 40%, about 50%, about 60%, about 70%, about 80%, about 90% or about 100%.

The level of lamin B1 protein and the level of lamin B1 farnesylation may be determined using methods known in the art, for example, immunological methods such as immunocytochemistry or Western blotting using labelled antibodies that bind the farnesyl group or lamin B1. Mass spectrometry techniques may also be used to quantify levels of lamin B1 and levels of farnesylation in a cell.

As used herein, the terms “treatment,” “treating,” and the like, refer to obtaining a desired pharmacologic and/or physiologic effect. The effect may be therapeutic in terms of a partial or complete cure for a disease or condition and/or adverse effect attributable to the disease or condition. These terms also cover any treatment of a condition or disease in a mammal, particularly in a human, and include: (a) inhibiting the disease or condition, i.e., arresting its development; or (b) relieving the disease or condition, i.e., causing regression of the disease or condition.

By “effective amount”, in the context of treating or preventing a disease or condition is meant the administration of an amount of active agent to a subject, either in a single dose or as part of a series or slow release system, which is effective for the treatment or prevention of that disease or condition. The effective amount will vary depending upon the health and physical condition of the subject and the taxonomic group of individual to be treated, the severity of the disease or condition, the formulation of the active agent or pharmaceutical composition, the assessment of the medical situation, and other relevant factors. The skilled person would be able to determine an effective amount of an active agent with consideration of, for example, a subject's age, weight, and clinical condition.

Disclosed herein is a method of identifying a subject who is likely to be responsive to treatment with a farnesyltransferase inhibitor (FTI), the method comprising detecting the level of lamin B1 in a sample from the subject, wherein an increase in the level of lamin B1 as compared to a reference indicates that the subject is likely to be responsive to a farnesyltransferase inhibitor (FTI).

The “sample” as used herein includes any biological specimen that may be extracted, untreated, treated, diluted or concentrated from a subject. A sample includes within its scope a collection of similar fluids, cells, or tissues (e.g., surgically resected tissue, biopsies, including fine needle aspiration), isolated from a subject, as well as fluids, cells, or tissues present within a subject. Any suitable methods for obtaining a biological sample can be employed; exemplary methods include, e.g., phlebotomy, swab (e.g., buccal swab), fine needle aspiration and forceps biopsy. The sample may be pooled from multiple aliquots.

A “reference”, “control”, “reference sample”, or “control sample”, as used herein, refers to a sample, cell, tissue, standard, or level that is used for comparison purposes. In one embodiment, a reference is obtained from a healthy and/or non-diseased part of the body (e.g., tissue or cells) of the same subject or individual. For example, healthy and/or non-diseased cells or tissue adjacent to the diseased cells or tissue. In another embodiment, a reference is obtained from an untreated tissue and/or cell of the body of the same subject or individual. In yet another embodiment, a reference is obtained from a healthy and/or non-diseased part of the body (e.g., tissues or cells) of an individual who is not the subject or individual. In another embodiment, a reference is obtained from an untreated tissue and/or cell of the body of an individual who is not the subject or individual. The reference may be population-average levels for a biomarker (e.g., lamin B1) in healthy cells or tissues.

As used herein, the term “increase” or “increased” with reference to a biomarker such as lamin B1 may refer to a statistically significant and measurable increase in the biomarker as compared to a reference. The increase may be an increase of at least about 10%, at least about 20%, at least about 30%, at least about 40%, at least about 50%, at least about 60%, at least about 70%, at least about 80%, at least about 90%, at least about 100%, at least about 2.5-fold, at least about 3-fold, at least about 3.5-fold, at least about 4-fold, at least about 4.5-fold, at least about 5-fold, or more.

In some embodiments, the subject is suffering from Adult-onset Autosomal Dominant Leukodystrophy (ADLD).

Disclosed herein is a method of treating a disease or condition in a subject associated with abnormal levels of lamin B1 protein, the method comprising: a) detecting the level of lamin B1 in a sample from the subject, wherein an increase in the level of lamin B1 as compared to a reference indicates that the subject is likely to be responsive to a farnesyltransferase inhibitor (FTI), and b) administering an effective amount of an FTI to the subject found likely to be responsive to an FTI.

In some embodiments, the disease or condition is Adult-onset Autosomal Dominant Leukodystrophy (ADLD).

Disclosed herein is a method of treating a disease or condition in a subject associated with abnormal levels of lamin B1 protein, the method comprising administering an effective amount of a farnesyltransferase inhibitor (FTI) to the subject.

In some embodiments, the disease or condition is characterized by abnormal farnesylation of lamin B1 protein. Abnormal farnesylation refers to a change in lamin B1 farnesylation which results in abnormally high levels of lamin B1. Such a change may be, for example, an increase in farnesylation efficiency.

In some embodiments, the disease or condition is characterized by accumulation of lamin B1 protein. In one embodiment, the disease or condition is Adult-onset Autosomal Dominant Leukodystrophy (ADLD).

Disclosed herein is a method of treating Adult-onset Autosomal Dominant Leukodystrophy (ADLD) in a subject, the method comprising administering an effective amount of a farnesyltransferase inhibitor (FTI) to the subject.

Also disclosed herein is a method of treating Adult-onset Autosomal Dominant Leukodystrophy (ADLD) in a subject, the method comprising: a) detecting the level of lamin B1 in a sample from the subject, wherein an increase in the level of lamin B1 as compared to a reference indicates that the subject is likely to be responsive to a farnesyltransferase inhibitor (FTI), and b) administering an effective amount of an FTI to the subject found likely to be responsive to an FTI.

In one embodiment, there is provided an FTI for use in treating a disease or condition in a subject associated with abnormal levels of lamin B1 protein. In one embodiment, there is provided an FTI for use in treating Adult-onset Autosomal Dominant Leukodystrophy (ADLD) in a subject.

In one embodiment, there is provided the use of an FTI in the manufacture of a medicament for treating a disease or condition in a subject associated with abnormal levels of lamin B1 protein. In one embodiment, there is provided the use of an FTI in the manufacture of a medicament for treating Adult-onset Autosomal Dominant Leukodystrophy (ADLD) in a subject.

The FTI as defined herein may be administered in a single dose or a series of doses. While it is possible for the active ingredient to be administered alone, it is preferable to present it as a composition, preferably as a pharmaceutical composition. The formulation of such compositions is well known to those skilled in the art. The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

In one embodiment, there is provided a pharmaceutical composition comprising an FTI and a pharmaceutically acceptable carrier.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, colouring agents, wetting or emulsifying agents, pH buffering agents, preservatives, and the like.

The carrier must be pharmaceutically “acceptable” in the sense of being compatible with the other ingredients of the composition and not injurious to the patient. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Representative pharmaceutically acceptable carriers include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

The pharmaceutical compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Suitable pharmaceutical compositions can be administered intravenously, subcutaneously, intramuscularly, or via any mucosal surface, e.g., orally, sublingually, buccally, sublingually, nasally, rectally, vaginally or via pulmonary route. In one embodiment, the mode of administration is oral. In one embodiment, the mode of administration is parenteral (e.g., intravenous, subcutaneous, intraperitoneal, intramuscular).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1 M and preferably 0.05 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin and/or by the maintenance of the required particle size. In specific embodiments, an FTI of the present disclosure may be conjugated to a vehicle for cellular delivery. In these embodiments, the FTI may be encapsulated in a suitable vehicle to either aid in the delivery of the FTI to target cells, to increase the stability of the FTI, or to minimize potential toxicity of the FTI. As will be appreciated by a skilled artisan, a variety of vehicles are suitable for delivering an FTI of the present disclosure. Non-limiting examples of suitable structured fluid delivery systems may include nanoparticles, liposomes, microemulsions, micelles, dendrimers and other phospholipid-containing systems. Methods of incorporating FTIs of the present disclosure into delivery vehicles are known in the art. Although various embodiments are presented below, it will be appreciate that other methods known in the art to incorporate an FTI of the disclosure into a delivery vehicle are contemplated.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. An FTI of the present disclosure can be administered on multiple occasions. Intervals between single dosages can be daily, weekly, monthly or yearly. Intervals can also be irregular as indicated by measuring blood levels of FTI in the patient. Alternatively, the FTI can be administered as a sustained release formulation, in which case less frequent administration is required. Dosage and frequency vary depending on the half-life of the FTI in the patient.

It is especially advantageous to formulate compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutically acceptable carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

In some embodiments, an effective amount of the FTI is administered orally or parenterally. In some embodiments, the FTI is administered orally in an amount of from 1 up to 1500 mg/kg daily, either as a single dose or subdivided into more than one dose, or more particularly in an amount of from 10 to 1200 mg/kg daily. In some embodiments, the FTI is administered orally in an amount of 100 mg/kg daily, 200 mg/kg daily, 300 mg/kg daily, 400 mg/kg daily, 500 mg/kg daily, 600 mg/kg daily, 700 mg/kg daily, 800 mg/kg daily, 900 mg/kg daily, 1000 mg/kg daily, 1100 mg/kg daily, or 1200 mg/kg daily.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In one embodiment, there is provided a pharmaceutical combination of an FTI and another therapeutic agent. The administration of the pharmaceutical combination of the present invention may result not only in a beneficial effect, e.g., an additive or synergistic therapeutic effect, for instance, with regard to alleviating, delaying progression of or inhibiting the symptoms, but also in further surprising beneficial effects. Such other effects may include fewer side effects, an improved quality of life or a decreased morbidity, compared with a monotherapy applying only one of the pharmaceutically active ingredients used in the combination of the present invention.

A further benefit of the invention is that lower doses of the active ingredients of the combination can be used. The dosages need not only be smaller but may also be applied less frequently, which may diminish the incidence or severity of side effects.

In particular, a therapeutically effective amount of each of the combination partners of the combination of the invention may be administered simultaneously or sequentially in any order, and the components may be administered separately or as a fixed combination.

The effective dosage of each of the combination partners employed in the combination of the invention may vary depending on the particular compound or pharmaceutical composition employed, the mode of administration, the condition being treated and the severity of the condition being treated. Thus, the dosage regimen of the combination of the invention is selected in accordance with a variety of factors including the route of administration and the renal and hepatic function of the patient. A person of ordinary skill can readily determine the effective amount of the single active ingredients required to alleviate, counter or arrest the progress of the condition.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features. Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### EXAMPLES

### Materials and Methods

### Cell Culture

NDF cell lines were provided by the Asian Skin Biobank (A*STAR) and cultured in NDF medium containing 15% fetal calf serum (Invitrogen, SH30071.03), 2 mM glutamine (Invitrogen, 25030081-P), 0.2 mM non-essential amino acids (Invitrogen, 10370088), and 50 U/ml Penicillin-Streptomycin (Invitrogen, 15140122) in minimum essential medium (Invitrogen, 10370088). Standard culture conditions were utilised (37 °C and 5% CO₂). Whenever cells were sub-cultured, 0.25% Trypsin with EDTA (Gibco, 25200056) was used and neutralised in dPBS (Cytiva, SH30028.02) with 10% fetal calf serum (Invitrogen, SV30160.03). Working concentration of 1 μg/ml of DOX (Clontech, 631311) and 5 μM of FTI-277 (obtained from A*SRL) were used in the indicated experiments.

Treatment with Statins and FTIs

NDFs with DOX-inducible LB1 were treated with 5 μM lovastatin, 5 μM of FTI-277, 2 μM of Lonafarnib and 1 μM of Tipifarnib.

### Western Blotting

Protein lysate extraction was performed using cOmplete™ Lysis-M EDTA-free kit (Roche, 4719964001), and cOmplete™ Protease Inhibitor Cocktail (Roche, 4693159001), following manufacturer's protocol, with the addition of 2% SDS (Promega, V6551), and 0.1 mM Dithiothreitol (Sigma, 646563). Quantification of proteins was done using Pierce™ Microplate BCA protein assay kit-Reducing Agent Compatible (Thermo Scientific™, 23252). SDS-PAGE was performed using 4-12% Bis-Tris Gel (Invitrogen) in NuPAGE™ MOPS SDS (Invitrogen, NP0001). The proteins were then transferred from the gel to nitrocellulose membranes (Bio-Rad, 1620115), and subsequently blocked in Intercept™ (PBS) Blocking Buffer (Li-COR, LIR. 927-70001) for 1 hr. Membranes were stained overnight at 4 °C with primary antibodies, washed with PBS with 0.1% tween-20 and stained with Odyssey Infrared-labelled secondary antibodies (LI-COR) in the dark at room temperature. Blots were visualised using a LI-COR Odyssey scanner. The integrated intensities obtained was normalised against the GAPDH or actin loading control signals and analysed in Microsoft® Excel.

### Antibodies

The following primary antibodies were used: v5 (Abcam; ab9137, and Invitrogen, R960-CUS); lamin B1 (ProteinTech, 66095-1 Ig); LA/C (Millipore; MAB3211, and ProteinTech, 10298-1-AP); GAPDH (Sigma; G9545, and ProteinTech; 10494-1-AP). Secondary antibodies for western blots are as follows: IRDYE 680 donkey anti-mouse IgG (Li-COR; 926-32222); IRDYE 800 donkey anti-mouse IgG (Li-COR; 926-32212); IRDYE 680 donkey anti-rabbit IgG (Li-COR; 926-68073); IRDYE 800 donkey anti-rabbit IgG (Li-COR; 926-32213); IRDYE 680 donkey anti-goat IgG (Li-COR; 926-32224); and IRDYE 800 donkey anti-goat IgG (Li-COR; 925-32214).

### EXAMPLE 1

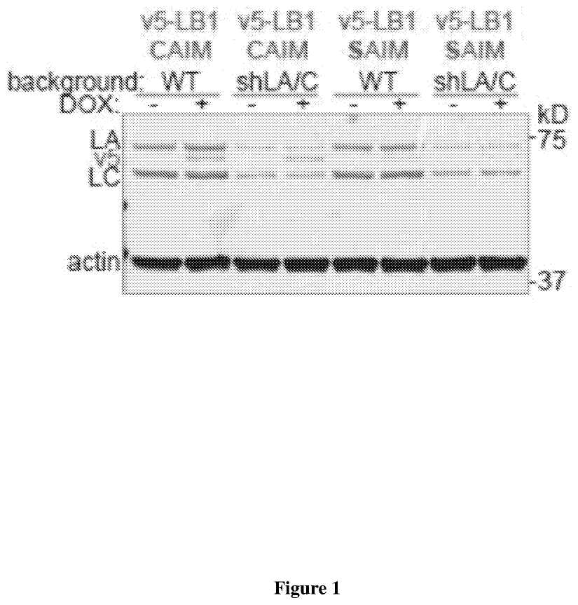

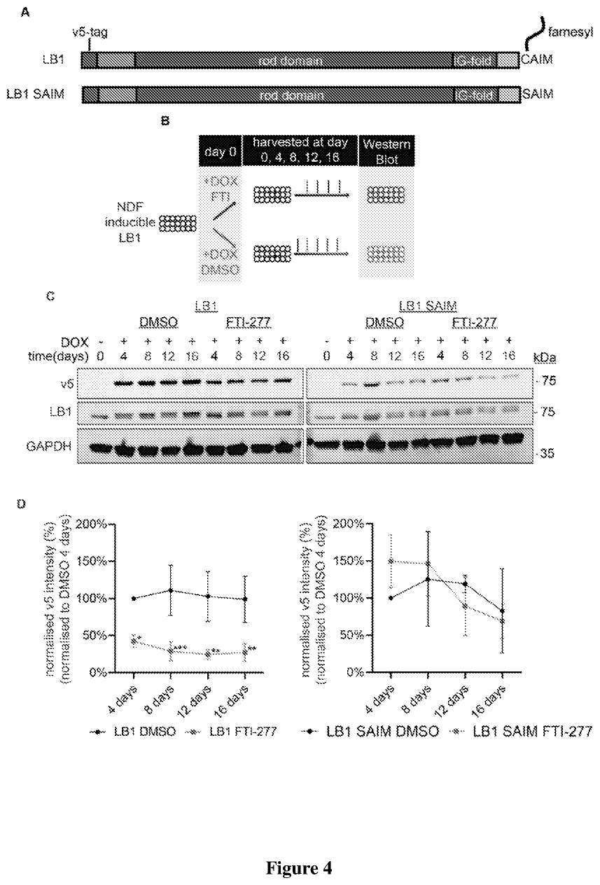

To investigate whether the farnesyl group may play a role in the aetiology of ADLD, a doxycycline-inducible system was used to ectopically express v5-tagged lamin B1 and a nonfarnesylatable mutant of lamin B1 (in which the farnesylatable C-terminal cysteine was changed to a serine, v5-LB1-CAIM->v5-LB1-SAIM), in normal and lamin A/C reduced (shLA/C) human primary neonatal dermal fibroblasts (NDF cells). Cells were treated with DOX in the presence of lovastatin and various FTIs over the course of 3 days (FIG. 1-3) or 16 days (FIG. 4). It was observed that the non-farnesylatable version of lamin B1 (v5-lamin B1 SAIM) does not accumulate as rapidly as wild type lamin B1 (v5-lamin B1 CAIM) protein (FIG. 1), suggesting that the farnesyl group increases the stability of lamin B1, and farnesylation of lamin B1 may be required for lamin B1 accumulation.

### EXAMPLE 2

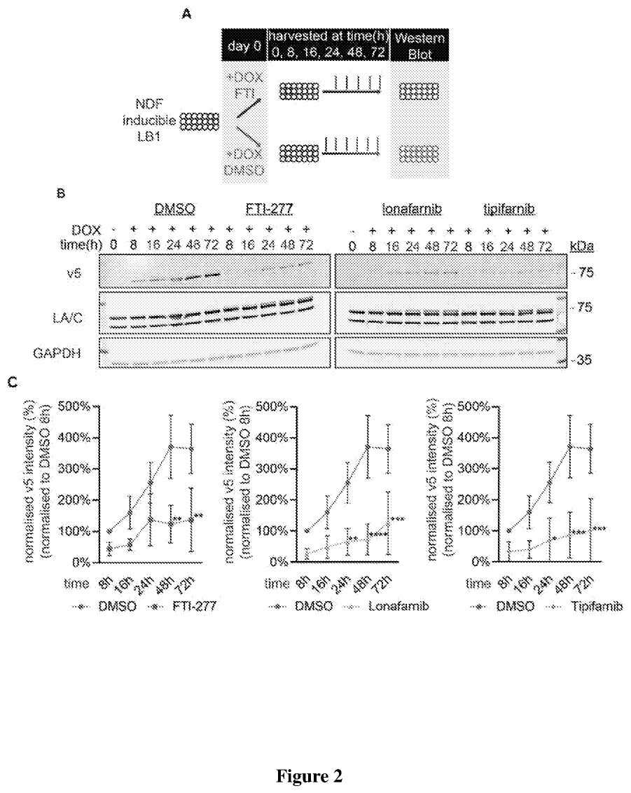

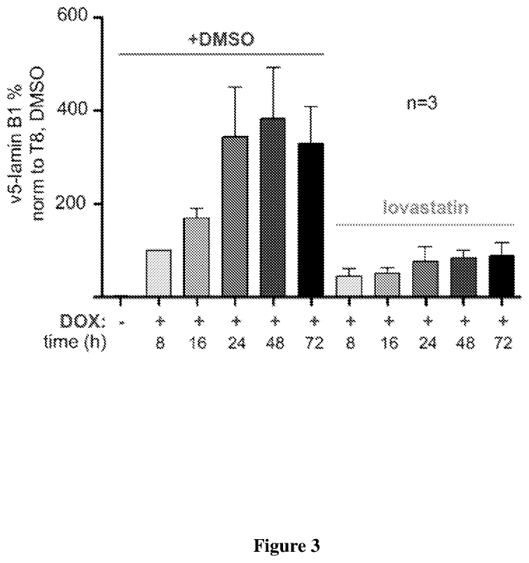

To investigate whether pharmacological prevention of farnesylation by farnesyltransferase inhibitors (FTI) would recapitulate these findings, v5-lamin B1 was expressed in the presence or absence of 3 different FTIs (FTI-277, Lonafarnib, Tipifarnib) and Lovastatin (a drug that reduces both isoprenoid and cholesterol synthesis). All 4 drugs significantly slowed the accumulation of lamin B1 over the course of 72 hours (FIGS. 2 and 3) and 16 days (FIG. 4). These results demonstrate that farnesylation plays a key role in regulating LB1 levels, and that this can be modulated by FTIs. Since ADLD patients have elevated LB1 levels, due to a duplication of the LMNB1 locus, and have no known treatment options, these findings strongly suggest that FTIs can be used to downregulate LB1 levels in these patients. Further, it may also be possible to repurpose FTIs to treat, prevent or delay the onset of diseases characterized by accumulation of permanently farnesylated proteins.

1. A method of inhibiting farnesylation of lamin B1 protein in a cell from a subject, the method comprising administering to the cell an effective amount of a farnesyltransferase inhibitor (FTI).

2. The method of claim 1, wherein the cell has abnormal levels of lamin B1 protein.

3. The method of claim 2, wherein the subject is suffering from Adult-onset Autosomal Dominant Leukodystrophy (ADLD).

4. The method of claim 1, to wherein inhibition of farnesylation of lamin B1 inhibits accumulation of lamin B1 protein.

5. The method of claim 1, to wherein the cell is a cell of the central or peripheral nervous system.

6. The method of claim 1, wherein the FTI is FTI- 277, lonafarnib or Tipifarnib.

7. The method of claim 1, wherein the subject is a human.

8. A method of inhibiting accumulation of lamin B1 protein in a cell from a subject, the method comprising administering to the cell an effective amount of a farnesyltransferase inhibitor (FTI).

9. A method of treating a disease or condition in a subject associated with abnormal levels of lamin B1 protein, the method comprising administering an effective amount of a farnesyltransferase inhibitor (FTI) to the subject.

10. The method of claim 9, wherein the disease or condition is characterized by accumulation or abnormal levels of lamin B1 protein.

11. The method of claim 9, or wherein the disease or condition is Adult-onset Autosomal Dominant Leukodystrophy (ADLD).

12. The method of claim 9, wherein the FTI is FTI-277, lonafarnib or Tipifarnib.

13. The method of claim 9, wherein the subject is a human.

14.-19. (canceled)
